(19) United States
(12) Patent Application Publication
(10) Pub. No.: US 2018/0214376 A1
(43) Pub. Date: Aug. 2, 2018

(54) SPHERICAL NUCLEIC ACID (SNA)-MEDIATED DELIVERY OF LIPID-COMPLEXES TO CELLS

(71) Applicant: EXICURE, INC., Skokie, IL (US)

(72) Inventor: David A. GILJOHANN, Chicago, IL (US)

(73) Assignee: Exicure, Inc., Skokie, IL (US)

(21) Appl. No.: 15/744,586

(22) PCT Filed: Jul. 14, 2016
(86) PCT No.: PCT/US2016/042291

§ 371 Date: Jan. 12, 2018

Related U.S. Application Data
(60) Provisional application No. 62/192,531, filed on Jul. 14, 2015.

(51) Int. Cl. A61K 9/127; A61K 31/713

(52) U.S. Cl. CPC A61K 9/1272; A61K 31/713

(57) ABSTRACT

The invention relates to spherical nucleic acids for the delivery of lipid nucleic acid particles. The lipid nucleic acid particles containing one or more therapeutic agents are coated with an oligonucleotide shell to enhance delivery.

### RELATED APPLICATIONS

This application claims the benefit under 35 U.S.C. § 119(e) of U.S. provisional application No. 62/192,531, filed Jul. 14, 2015, which is incorporated by reference herein in its entirety.

### BACKGROUND OF THE INVENTION

Currently, nucleic acid transfection commonly relies on lipid-mediated delivery. Additionally, many types of molecules are liposome-encapsulated for delivery. Major divisions of lipid-mediated delivery include: encapsulation of the nucleic acid within liposomes and lipid-nucleic acid complexes (“lipoplexes”), most commonly containing cationic lipids. Cationic and non-cationic lipid approaches have differences in toxicity, efficiency, and stability. Neutral lipid liposomes are non-toxic and stable in serum. Toxicity has been a limitation of lipoplexes containing cationic lipids.

### SUMMARY OF THE INVENTION

In some aspects the invention is a spherical nucleic acid (SNA). The SNA has a lipid nanoparticle composed of a liposome or lipoplex complex containing a therapeutic agent, an oligonucleotide shell comprised of oligonucleotides positioned on the exterior of the lipid nanoparticle. At least 10% of the oligonucleotides are attached to the lipid nanoparticle through a lipid anchor group. In some embodiments at least 20%, 40%, 50%, 80%, 90%, or 100% of the oligonucleotides are attached to the lipid nanoparticle through a lipid anchor group. In other embodiments the oligonucleotides of the oligonucleotide shell are oriented radially outwards.

The lipid nanoparticle in some embodiments is a liposome encapsulating a nucleic acid, a lipoplex complex containing cationic lipids, or a lipoplex complex containing non-cationic lipids. In other embodiments the lipid nanoparticle comprises a lipid selected from the group consisting of a neutral lipid, a zwitterionic lipid, a cationic lipid, and an anionic lipid. In yet other embodiments the lipid nanoparticle is PEGylated.

The therapeutic agent may be any type of therapeutic or active agent that is deliverable in a lipid carrier such as a liposome. In some embodiments the therapeutic agent is nucleic acids, small molecules, proteins, gases (e.g. NO), dyes, vitamins, nutrients, antibiotics, antifungals, antivirals, chemotherapeutic agents, steroids, hormones, magnetic or paramagnetic particles, a pro-drug, a water soluble therapeutic agent, a water insoluble therapeutic agent, and therapeutic proteins associated with endosomal storage diseases. In some embodiments the nucleic acids are selected from the group consisting of plasmids, antisense oligonucleotides, immunostimulatory oligonucleotides, immunoinhibitory oligonucleotides, mRNA, long ncRNA, siRNA, and miRNA. In other embodiments the therapeutic agent is a nucleic acid complexed with a protein, a polymer, or a carrier such as protamine.

The lipid nanoparticle may, in some embodiments, comprise a surface active agent associated with the lipid surface, such as an agent selected from aptamers, antibodies, proteins, peptides, lipid derivatives, small molecules, and magnetic or paramagnetic particles.

The oligonucleotides of the oligonucleotide shell may be any type of oligonucleotide. For instance, in some embodiments, the oligonucleotides are comprised of single-stranded or double-stranded DNA oligonucleotides or mixtures thereof, single-stranded or double-stranded RNA oligonucleotides or mixtures thereof or chimeric RNA-DNA oligonucleotides. In other embodiments the oligonucleotides of the oligonucleotide shell are comprised of combinations of single-stranded or double-stranded DNA, RNA, or chimeric RNA-DNA oligonucleotides.

In some embodiments the oligonucleotides of the oligonucleotide shell have structurally identical oligonucleotides. In other embodiments the oligonucleotides of the oligonucleotide shell have at least two structurally different oligonucleotides. The oligonucleotides of the oligonucleotide shell may have 2-10 different nucleotide sequences.

In some embodiments the oligonucleotides of the oligonucleotide shell are functional oligonucleotides such as CpG-motif containing oligonucleotides, antisense oligonucleotides, or therapeutic oligonucleotides. In yet other embodiments the oligonucleotides are non-functional oligonucleotides. In other embodiments the oligonucleotides of the oligonucleotide shell do not comprise CpG-motif containing oligonucleotides, an immuno-inert oligonucleotides or oligonucleotides having a length of 8-200 nucleotides and including at least one GGG. The oligonucleotides have random nucleic acid sequences in other embodiments.

The oligonucleotide shell may be a dense shell. In some embodiments the oligonucleotide shell has a density of 5-1,000 oligonucleotides per SNA, 100-1,000 oligonucleotides per SNA, or 500-1,000 oligonucleotides per SNA.

The oligonucleotides of the oligonucleotide shell may have at least one internucleoside phosphorothioate linkage. In some embodiments the oligonucleotides of the oligonucleotide shell do not have an internucleoside phosphorothioate linkage. In other embodiments the oligonucleotides of the oligonucleotide shell have all internucleoside phosphorothioate linkages.

The oligonucleotides of the oligonucleotide shell have any length. For instance oligonucleotides of the oligonucleotide shell may have a length of 10 to 100 nucleotides, 10 to to 80 nucleotides, 10 to 50 nucleotides or 10 to 30 nucleotides.

In some embodiments at least 25 percent of the oligonucleotides of the oligonucleotide shell have 5′-termini exposed to the outside surface of the SNA. In other embodiments all of the oligonucleotides of the oligonucleotide shell have 5′ termini exposed to the outside surface of the SNA. In yet other embodiments at least 25 percent of the oligonucleotides of the oligonucleotide shell have 3′-termini exposed to the outside surface of the SNA. All of the oligonucleotides of the oligonucleotide shell have 3′-termini exposed to the outside surface of the SNA in other embodiments.

In some embodiments the SNA is a self-assembling nanostructure.

A method for delivering a therapeutic agent to a subject by administering to a subject a SNA as described herein, in an effective amount to deliver the therapeutic agent to the subject is provided in other aspects. In some embodiments the therapeutic agent is delivered to a cell of the subject. In other embodiments the therapeutic agent is delivered to endosomes through scavenger receptors.

In other aspects the invention is a composition for use in the treatment of disease, comprising any of the SNA described and claimed herein.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### DETAILED DESCRIPTION OF THE INVENTION

Spherical nucleic acids (SNAs) consist of densely packed, radially oriented nucleic acids. This architecture gives them unique properties, enabling cellular uptake of SNAs mediated via scavenger receptors. Cellular uptake of SNAs is fast and efficient and leads to endosomal accumulation.

Spherical nucleic acids (SNAs) are a class of well-defined macromolecules, formed by organizing nucleic acids radially around an inorganic metallic nanoparticle core (Mirkin C A, Letsinger R L, Mucic R C, & Storhoff J J (1996) A DNA-based method for rationally assembling nanoparticles into macroscopic materials. Nature 382(6592):607-609.). These structures exhibit the ability to enter cells without the need for auxiliary delivery vehicles or transfection reagents by engaging class A scavenger receptors (SR-A) and lipid rafts (Patel PC, et al. (2010) Scavenger receptors mediate cellular uptake of polyvalent oligonucleotide-functionalized gold nanoparticles. Bioconjugate chemistry 21(12):2250-2256.). Once inside the cell, the nucleic acid components of traditional SNAs resist nuclease degradation, leading to longer intracellular lifetimes. Moreover, SNAs, due to their multi-functional chemical structures, have the ability to bind their targets in a multivalent fashion (Choi C H, Hao L, Narayan S P, Auyeung E, & Mirkin C A (2013) Mechanism for the endocytosis of spherical nucleic acid nanoparticle conjugates. Proceedings of the National Academy of Sciences of the United States of America 110(19):7625-7630; Wu X A, Choi C H, Zhang C, Hao L, & Mirkin C A (2014) Intracellular fate of spherical nucleic acid nanoparticle conjugates. Journal of the American Chemical Society 136(21):7726-7733).

It has been discovered herein that SNA formulation technology can be utilized to enhance delivery to lipid based delivery systems. SNAs have been developed according to the invention which incorporate lipid nanoparticles in a densely packed oligonucleotide shell. These unique molecules can be used to efficiently deliver any type of therapeutic or diagnostic reagent to a cell, and in particular to endosomes. Following liposome encapsulation or lipoplexing of a therapeutic molecule or molecules, the liposome or lipoplex can be functionalized into an SNA by inserting lipid-conjugated nucleic acids to its external surface. The resulting SNAs will contain the molecule of interest, lipids, and the outer radially oriented nucleic acids. Molecules packaged in the aforementioned SNAs will be taken up into cells via scavenger receptor-mediated endocytosis, resulting in efficient and fast endosomal accumulation characteristic of other SNAs. Functionalizing the liposome/lipoplex as an SNA changes the route of uptake. Functionalizing the liposome/lipoplex as an SNA increases the efficiency, kinetics, or endosomal accumulation of the liposome/lipoplex. By functionalizing the surface of a liposome/lipoplex, containing therapeutic cargo, into a SNA, the route of cellular delivery is directed through scavenger receptors, enhancing endosomal uptake in vitro and in vivo.

The nanostructures of the invention are typically composed of lipid nanoparticles having a therapeutic agent incorporated therein and a shell of oligonucleotides, which is formed by arranging oligonucleotides such that they point radially outwards from the core. A hydrophobic (e.g. lipid) anchor group attached to either the 5′- or 3′-end of the oligonucleotide, depending on whether the oligonucleotides are arranged with the 5′- or 3′-end facing outward from the core preferably is used to embed the oligonucleotides in the lipid nanoparticle. The anchor acts to drive insertion into the lipid nanoparticle and to anchor the oligonucleotides to the lipids.

The lipid nanoparticle can be constructed from a wide variety of lipids known to those in the art to produce a liposome or lipoplex. A liposome is a structure composed of at least one lipid bilayer membrane that encloses an internal compartment. Liposomes may be characterized according to the membrane type and size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

A lipoplex, is a type of lipid nanoparticle which specifically incorporates nucleic acids in a lipid-nucleic acid complex. Lipoplexes, also referred to as nucleic acid lipid particles typically contain a cationic lipid or a non-cationic lipid and optionally a sterol and/or a lipid that prevents aggregation of the particle (e.g., a PEG-lipid conjugate). In some instances the lipoplex contains a cationic lipid, a non-cationic lipid, a sterol and a lipid.

Other lipids may be included in the lipid nanoparticle for a variety of purposes, such as to prevent lipid oxidation or to attach ligands onto lipid nanoparticle surface. Any of a number of lipids may be present, including amphipathic, neutral, cationic, and anionic lipids. Such lipids can be used alone or in combination. Additional components that may be present in a lipid nanoparticle include bilayer stabilizing components such as polyamide oligomers, peptides, proteins, detergents, lipid-derivatives, such as PEG coupled to phosphatidylethanolamine and PEG conjugated to ceramides. The lipid nanoparticles may also include one or more of a second amino lipid or cationic lipid, a neutral lipid, a sterol, and a lipid selected to reduce aggregation of lipid particles during formation, which may result from steric stabilization of particles which prevents charge-induced aggregation during formation. As used herein, the term “amino lipid” is meant to include those lipids having one or two fatty acid or fatty alkyl chains and an amino head group (including an alkylamino or dialkylamino group) that may be protonated to form a cationic lipid at physiological pH.

The particles of the present in lipid nanoparticle may have a mean diameter of about 50 nm to about 150 nm, more typically about 60 nm to about 130 nm, more typically about 70 nm to about 110 nm, most typically about 70 nm to about 90 nm.

In one embodiment, the lipid to drug ratio (mass/mass ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or about 6:1, 7:1, 8:1, 9:1, 10:1, 11:1, 12:1, or 33:1.

The lipid nanoparticle may include a cationic lipid. The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate, or a mixture thereof.

Other cationic lipids, which carry a net positive charge at about physiological pH, in addition to those specifically described above, may also be included in the lipid nanoparticle. Such cationic lipids include, but are not limited to, N,N-dioleyl-N,N-dimethylammonium chloride (“DODAC”); N-(2,3-dioleyloxy)propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3.beta.-(N-(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”).

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleylphosphatidylcholine.

The oligonucleotide shell can be constructed from a wide variety of nucleic acids including, but not limited to: single-stranded deoxyribonucleotides, ribonucleotides, and other single-stranded oligonucleotides incorporating one or a multiplicity of modifications known to those in the art, double-stranded deoxyribonucleotides, ribonucleotides, and other double-stranded oligonucleotides incorporating one or a multiplicity of modifications known to those in the art, oligonucleotide triplexes incorporating deoxyribonucleotides, ribonucleotides, or oligonucleotides that incorporate one or a multiplicity of modifications known to those in the art.

For instance, the oligonucleotides may be functional oligonucleotides or therapeutic oligonucleotides. Functional oligonucleotides include but are not limited to mRNA, ncRNA (long ncRNA, siRNA, miRNA, ASO, ORN, etc.), DNA (plasmid, ASO, ODN, etc.). These oligonucleotides may or may not be pre-complexed with proteins, polymers, or carriers including protamine.

Therapeutic agents are incorporated into the lipid nanoparticles. These include but are not limited to small molecules, proteins, nucleic acids, gases (e.g. NO), dyes, vitamins, nutrients, antibiotics, antifungals, and antivirals, chemotherapeutic agents, steroids, hormones, magnetic or paramagnetic particles, encapsulated therapeutic drugs, prodrugs or molecules, water soluble or water insoluble molecules, and proteins, including those that function in the endosome, especially the ones associated with endosomal storage diseases.

Lipid composition may include neutral, zwitterionic, cationic, and anionic compositions. In some embodiments these compositions may or may not be PEGylated.

The liposome/lipoplex may also be constructed to contain other surface elements including but not limited to: aptamers, antibodies, proteins, peptides, lipid derivatives; small molecules, and magnetic/paramagnetic particles. These may be used for various purposes. In some embodiments the complex to target specific cells/tissues, inducing antigen-specific immune responses in vivo and in vitro.

The terms “oligonucleotide” and “nucleic acid” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymidine (T) or uracil (U)) or a substituted purine (e.g., adenine (A) or guanine (G)). Thus, the term embraces both DNA and RNA oligonucleotides. The terms shall also include polynucleosides (i.e., a polynucleotide minus the phosphate) and any other organic base containing polymer. Oligonucleotides can be obtained from existing nucleic acid sources (e.g., genomic or cDNA), but are preferably synthetic (e.g., produced by nucleic acid synthesis). The term “nucleoside” includes bases which are covalently attached to a sugar moiety, preferably ribose or deoxyribose. The term “nucleotide” includes nucleosides which further comprise a phosphate group or a phosphate analog.

Oligonucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, oligonucleotides associated with the invention may only contain modified or unmodified sugar moieties, while in other instances, oligonucleotides contain some sugar moieties that are modified and some that are not.

In some instances, modified nucleotides include sugar- or backbone-modified ribonucleotides or deoxyribonucleotides. Modified ribo or deoxyribonucleotides can contain a non-naturally occurring base such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH₂, NHR, NR₂), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. The sugar moiety can also be a hexose.

The term “hydrophobic modifications” refers to modification of bases such that overall hydrophobicity is increased and the base is still capable of forming close to regular Watson-Crick interactions. Non-limiting examples of base modifications include 5-position uridine and cytidine modifications like phenyl, 4-pyridyl, 2-pyridyl, indolyl, and isobutyl, phenyl (C₆H₅OH); tryptophanyl (C₈H₆N)CH₂CH(NH₂)CO), Isobutyl, butyl, aminobenzyl; phenyl; and naphthyl.

In some aspects, oligonucleotides of the invention comprise 3′ and 5′ termini. The 3′ and 5′ termini of an oligonucleotide can be substantially protected from nucleases, for example, by modifying the 3′ or 5′ linkages (e.g., U.S. Pat. No. 5,849,902 and WO 98/13526). Oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²⁻), hydrogen phosphonate, or phosphoramidite). “Blocking groups” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleotide can comprise a modified sugar moiety. The 3′ terminal nucleotide comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

In some aspects, oligonucleotides can comprise both DNA and RNA.

In some aspects, at least a portion of the oligonucleotides are linked by a substitute linkage, e.g., a phosphorothioate linkage. The presence of substitute linkages can improve pharmacokinetics due to their higher affinity for serum proteins.

The oligonucleotides are preferably in the range of 6 to 100 bases in length. However, nucleic acids of any size greater than 6 nucleotides (even many kb long) are useful. Preferably the nucleic acid is in the range of between 8 and 100 and in some embodiments between 8 and 50, 8 and 40, 8 and 30, 6 and 50, 6 and 40, or 6 and 30 nucleotides in size.

The surface density of the oligonucleotides may depend on the size and type of the core and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 100 oligonucleotides per particle will be adequate to provide stable core-oligonucleotide conjugates. Preferably, the surface density is at least 200, 300, 400, 500, 600, 700, 800, 900, 1,000, 1,200, 1,400, 1,600, 1,800, or 2,000 oligonucleotides per particle.

The oligonucleotide shell consists of oligonucleotides densely arranged radially around the core and having a lipid anchor. The lipid anchor consists of a hydrophobic group that enables insertion and anchoring of the nucleic acids to the lipid membrane.

Aspects of the invention relate to delivery of SNAs to a subject for therapeutic and/or diagnostic use. The SNAs may be administered alone or in any appropriate pharmaceutical carrier, such as a liquid, for example saline, or a powder, for administration in vivo. They can also be co-delivered with larger carrier particles or within administration devices. The SNAs may be formulated. The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients. In some embodiments, SNAs associated with the invention are mixed with a substance such as a lotion (for example, aquaphor) and are administered to the skin of a subject, whereby the SNAs are delivered through the skin of the subject. It should be appreciated that any method of delivery of nanoparticles known in the art may be compatible with aspects of the invention.

For use in therapy, an effective amount of the SNAs can be administered to a subject by any mode that delivers the SNAs to the desired cell. Administering pharmaceutical compositions may be accomplished by any means known to the skilled artisan. Routes of administration include but are not limited to oral, parenteral, intramuscular, intravenous, subcutaneous, mucosal, intranasal, sublingual, intratracheal, inhalation, ocular, vaginal, dermal, rectal, and by direct injection.

The SNAs described herein may be used in some embodiments for treating exosome mediated disease. A method of diagnosing, preventing, treating, or managing a disease or bodily condition may involve, for example, administering to a subject a therapeutically-effective amount of a SNA and allowing the SNA to be taken up by exosomes. Any disease that is mediated by exosomes can be effectively treated by delivering an SNA that includes one or more therapeutic agents for the treatment of that disease. Exosome mediated disease include for instance cancer and inflammatory diseases.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A spherical nucleic acid (SNA), comprising
a lipid nanoparticle comprising a liposome or lipoplex complex containing a therapeutic agent, an oligonucleotide shell comprised of oligonucleotides positioned on the exterior of the lipid nanoparticle, wherein at least 10% of the oligonucleotides are attached to the lipid nanoparticle through a lipid anchor group.

2. The SNA of claim 1, wherein the oligonucleotides of the oligonucleotide shell are oriented radially outwards.

3. The SNA of claim 1, wherein the lipid nanoparticle is a liposome encapsulating a nucleic acid.

4. The SNA of claim 1, wherein the lipid nanoparticle is a lipoplex complex containing cationic lipids.

5. The SNA of claim 1, wherein the lipid nanoparticle is a lipoplex complex containing non-cationic lipids.

6. The SNA of claim 1, wherein the lipid nanoparticle comprises a lipid selected from the group consisting of a neutral lipid, a zwitterionic lipid, a cationic lipid, and an anionic lipid.

7. The SNA of claim 1, wherein at least 20%, 40%, 50%, 80%, 90% or 100% of the oligonucleotides are attached to the lipid nanoparticle through a lipid anchor group.

8. (canceled)

9. The SNA of claim 1, wherein the therapeutic agent is a nucleic acid, small molecules, proteins, gases (e.g. NO), dyes, vitamins, nutrients, antibiotics, antifungals, antivirals, chemotherapeutic agents, steroids, hormones, magnetic or paramagnetic particles, a pro-drug, a water soluble therapeutic agent, a water insoluble therapeutic agent, and therapeutic proteins associated with endosomal storage diseases.

10. The SNA of claim 9, wherein the nucleic acids are selected from the group consisting of plasmids, antisense oligonucleotides, immunostimulatory oligonucleotides, immunoinhibitory oligonucleotides, mRNA, long ncRNA, siRNA, and miRNA.

11. The SNA of claim 1, wherein the therapeutic agent is a nucleic acid complexed with a protein, a polymer, or a carrier such as protamine.

12. The SNA of claim 1, wherein the lipid nanoparticle comprises a surface active agent associated with the lipid surface.

13. (canceled)

14. The SNA of claim 1, wherein the oligonucleotides of the oligonucleotide shell are comprised of single-stranded or double-stranded DNA oligonucleotides or mixtures thereof, or
wherein the oligonucleotides of the oligonucleotide shell are comprised of single-stranded or double-stranded RNA oligonucleotides or mixtures thereof.

15. (canceled)

16. The SNA of claim 1, wherein the oligonucleotides of the oligonucleotide shell are comprised of chimeric RNA-DNA oligonucleotides.

17. The SNA of claim 1, wherein the oligonucleotides of the oligonucleotide shell are comprised of combinations of single-stranded or double-stranded DNA, RNA, or chimeric RNA-DNA oligonucleotides.

18. The SNA of claim 1, wherein the oligonucleotides of the oligonucleotide shell have structurally identical oligonucleotides.

19. The SNA of claim 1, wherein the oligonucleotides of the oligonucleotide shell have at least two structurally different oligonucleotides.

20. (canceled)

21. The SNA of claim 1, wherein the oligonucleotides of the oligonucleotide shell comprise CpG-motif containing oligonucleotides.

22. The SNA of claim 1, wherein the oligonucleotides of the oligonucleotide shell do not comprise CpG-motif containing oligonucleotides, an immunoinert oligonucleotides or oligonucleotides having a length of 8-200 nucleotides and including at least one GGG.

23. The SNA of claim 1, wherein the oligonucleotides are nonfunctional oligonucleotides.

24. The SNA of claim 1, wherein the oligonucleotides have random nucleic acid sequences.

25. The SNA of claim 1, wherein the oligonucleotide shell has a density of 5-1,000 ligonucleotides per SNA.

26. (canceled)

27. (canceled)

28. The SNA of claim 1, wherein the oligonucleotides of the oligonucleotide shell have at least one internucleoside phosphorothioate linkage.

29. (canceled)

30. (canceled)

31. The SNA of claim 1, wherein the oligonucleotides of the oligonucleotide shell have a length of 10 to 100 nucleotides.

32.-34. (canceled)

35. The SNA of claim 1, wherein at least 25 percent of the oligonucleotides of the oligonucleotide shell have 5′-termini exposed to the outside surface of the SNA.

36. (canceled)

37. The SNA of claim 1, wherein at least 25 percent of the oligonucleotides of the oligonucleotide shell have 3′-termini exposed to the outside surface of the SNA.

38. (canceled)

39. The SNA of claim 1, wherein the SNA is a self-assembling nanostructure.

40. A method for delivering a therapeutic agent to a subject, comprising administering to a subject a SNA of claim 1, in an effective amount to deliver the therapeutic agent to the subject.

41. The method of claim 40, wherein the therapeutic agent is delivered to a cell of the subject.

42. The method of claim 41, wherein the therapeutic agent is delivered to endosomes through scavenger receptors.

43. A composition for use in the treatment of disease, comprising the SNA of claim 1.
